(19) United States
(12) Patent Application Publication
(10) Pub. No.: US 2003/0064500 A1
(43) Pub. Date: Apr. 3, 2003

(54) Method for continuous cultivation of microorganisms of the genus eremothecium

(76) Inventors: Stephan Freyer, Neustadt (DE); Henning Althofer, Limburgerhof (DE); Klaus-Peter Stahmann, Julich (DE); Andreas Wiesenberg, Jena (DE); Cornelia Gaetgens, Juelich-Selgersdorf (DE); Hermann Sahn, Julich (DE)

Correspondence Address: Nixon & Vanderhye, 1100 North Glebe Road 8th Floor, Arlington, VA, 22201-4714, US

(21) Appl. No.: 10/149,380

(22) PCT Filed: Dec. 14, 2000
(86) PCT No.: PCT/EP00/12696

(30) Foreign Application Priority Data
Dec. 16, 1999 (DE) 199607036

(51) Int. Cl. C12N 1/20

(52) U.S. Cl. 435/252.1

(57) ABSTRACT

The invention relates to a method for the continuous cultivation of a microorganism of the genus Eremothecium, to the products manufactured with said microorganism and to the utilization thereof.

The present invention relates to a process for the continuous culture of microorganisms of the genus Eremothecium and to products prepared by this process.

A large number of processes for culturing yeasts or fungi which grow in a yeast-like fashion, such as, for example, Saccharomyces or Candida species, have been described in the literature. This applies both to batch or fed-batch processes and to continuous fermentations. Advantages of the continuous biotechnological processes which must be mentioned in this context are, in particular, the reduction of idle time, a productivity which is higher in many instances, and the continuous recovery of products.

The filamentous fungus Eremothecium gossypii (synonym: Ashbya gossypii; Kurtzman, C. P., J. Ind. Microbiol., 1995, 14:523-530) has previously been of biotechno-logical importance owing to its ability to produce riboflavin (Vandamme E. J., J. Chem. Tech. Biotechnol., 1992, 53:313-327). Thus, the production of riboflavin by fermentation of Eremothecium gossypii and of the closely related fungus Eremothecium ashybi is known (The Merck Index, Windholz et al., eds. Merck & Co., 1983, pages 1183; Bacher A. et al., Angew. Chem., 1969, page 393). However, owing to the fact that the fungus Eremothecium gossypii has a better genetic stability than Eremothecium ashybi, E. gossypii is preferred for the industrial production of metabolites (Demain, A. L., Annu. Rev. Microbiol., 1972, 26:369-388).

However, the processes which have hitherto been described for the culture of filamentous fungi of the genus Eremothecium are exclusively batchwise culture processes, i.e. the cells are grown in what are known as batch or fed-batch cultures.

In general, growing filamentous fungi in continuous culture is very complicated and problematic. Examples which may be mentioned in this context are, mainly, the copious formation of mycelium during the culturing. While continuous processes have been described for Aspergillus or Penicillium species by Wongwicharn et al. (Biotechnol. Bioeng., 1999, 65 (4): 416-424) and Christensen et al. (J. Biotechnol., 1995, 42 (2); 95-107), these processes remain irrelevant for a large-scale production process. An industrially exploited process for the continuous culturing of a filamentous fungus has only been described for Fusarium graminearum (Trinci, A. P., Microbiology, 1994, 140 (Pt 9): 2181-2188).

Such a continuous process has not been available as yet for the fungus of the genus Eremothecium which displays filamentous growth. However, this would be very desirable, in particular with a view to the increasingly economical interest in this fungus.

The present invention relates to a process for culturing a microorganism of the genus Eremothecium in which the cells are cultured in a continuous fermentation at flow rates ranging from greater zero to 0.8 h⁻¹.

In accordance with the invention, the process is preferably carried out at flow rates ranging from 0.001 to 0.8 h⁻¹. Preferred flow rates range ranging from 0.01 to 0.7 h⁻¹, especially preferred flow rates from 0.05 to 0.6 h⁻¹.

In the traditional manner, a batch-operated fermentation is distinguished by the fact that the microorganism starts from a lag phase and undergoes a log phase, a stationary phase and a death phase. This means that different culture conditions prevail at any given point in time, that is to say that the culture conditions change all the time. In contrast, a continuous fermentation is distinguished by the fact that a state of equilibrium (flow equilibrium) establishes so that identical culture conditions prevail permanently. Values ranging from 0.15 to 0.25 h⁻¹ have been described in the literature for the maximum specific growth rate in the continuous culture of filamentous fungi such as, for example, Penicillium or Fusarium species on glucose-containing medium (Christensen et al., J. Biotechnol., 1995, 42: 95-107; Wiebe et al., Microbiology, 1994, 140: 3015-3021). Surprisingly, the process according to the invention is distinguished by the fact that a maximum specific growth rate of a microorganism of the genus Eremothecium ranging from 0.5-0.6 h⁻¹, in particular 0.55 h⁻¹, is permanently achieved.

This rapid growth constitutes a further advantage of the process according to the invention, which is of particular economic relevance, in particular regarding the industrial-scale application.

The present invention furthermore relates to a process in which the flow rate is alternatingly adjusted to constant higher or lower values at defined intervals.

In this context, the interval between the alternation between the flow rates is to be selected in such a way that, firstly, the stability of the system (establishment of the steady state) and, secondly, a desired maximum productivity are ensured. However, this depends on the intended aim of the culture process which is carried out.

Examples of the higher flow rates which are set constantly are values ranging from 0.1 to 0.8 h⁻¹, preferably 0.12 to 0.5 h⁻¹. Values of lower flow rates set constantly range, for example, from 0.01 to 0.2 h⁻¹, with ranges of from 0.02 to 0.1 h⁻¹ being preferred and a flow rate of approx. 0.05 h⁻¹ being especially preferred.

In a particular embodiment of the present invention, the flow rate is lowered to a value of approx. 0.05 h⁻¹ after the steady state has established at a flow rate of approx. 0.16 h^{-1.} During the process of establishing the new flow equilibrium, the dry biomass (DBM) formed is determined. An increase in dry biomass from 1.63 g/l to 3.93 g/l is recorded within approximately 8 hours. The consequence of lowering the flow rate is that the dry biomass markedly exceeds the value of 3.05 g of DMB/l, which establishes during the steady state. In further variants of the abovementioned method, the flow rate is similarly reduced starting from an initial flow rate of approximately 0.20 h⁻¹ or approximately 0.30 h⁻¹.

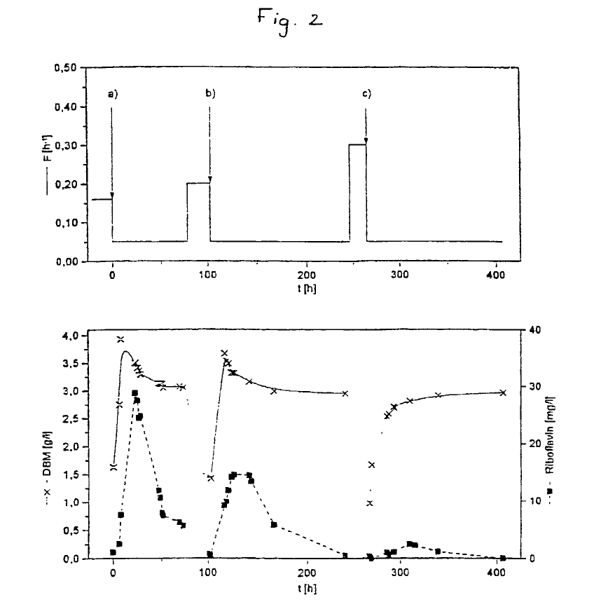

Surprisingly, this process is particularly suitable according to the invention for increasing the riboflavin production during the continuous culture of the fungus. A graphic representation of the dry biomass and riboflavin concentration curves is shown in FIG. 2.

It is a further advantage of the process according to the invention that the cells are stably cultured continuously over a period of several weeks, preferably 2 to 8 weeks, especially preferably 2 to 4 weeks, in particular more than 2.5 weeks (corresponding to more than 400 hours).

In accordance with the invention, at flow rates of greater zero to 0.20 h⁻¹, preferably 0.01 to 0.20 h⁻¹, the cells grow predominantly as single cells and/or spores develop. During this stage of the culture, product formation is predominantly growth-independent.

The process according to the invention is further distinguished by the fact that the microorganism predominantly grows as a hyphate mycelium and/or loose agglomerations of this mycelium in the form of mycelial pellets are formed at flow rates ranging from ≧0.20-0.70 h⁻¹. In this context, the mycelial pellets attain a size of 1 to 3 mm in diameter. During this stage of the culture, growth-dependent product formation predominates.

The formation of mycelial pellets proves to be very advantageous since these pellets sediment relatively rapidly and quantitatively and thus make possible the simple removal of the biomass from the culture medium without unduly complicated technical equipment and without further process steps. As a consequence, the subsequent work-up of the desired metabolites is simplified.

A fungus of the species Eremothecium gossypii is preferably employed for this purpose in the process according to the invention. In an especially preferred embodiment of the present invention, a microorganism which is genetically modified over the wild type Eremothecium gossypii (synonym: Ashbya gossypii) ATCC 10895 is employed.

Genetic modifications are understood as meaning, in accordance with the invention, natural, i.e. spontaneously occurring, or artificially generated mutations. Artificially generated mutations can be caused for example by treating the fungus with mutagenic agents or by irradiation, in particular UV radiation. These mutations are also termed undirected mutations.

The present invention furthermore encompasses genetically modified microorganisms which can be generated in a directed fashion by recombinant methods. In general, mutations encompass substitutions, additions, deletions, exchanges or insertions of one or more nucleotide residues, which can be of homologous or heterologous origin. These mutations can have an effect on gene expression or on the activity of the gene product, which may be reduced or increased. The mutations can be encoded chromosomally or be present in a multiple copy number on what are known as extrachromosomal gene structures (vectors). Also included in accordance with the invention, besides genetic modifications on a single gene, are simultaneous modifications on a plurality of genes within an organism. For example, the metabolism of the microorganism can be directed predominantly toward a desired product in this manner (metabolic design).

In accordance with the invention, homologous and/or heterologous products, for example polysaccharases, lipases or proteases and sugars or organic acids, inter alia amino acids, are formed by the present process. Also, the production of heterogeneous gene products, which consist of a mixture of homologous and heterologous components, such as, for example, fusion proteins, is possible.

The present invention furthermore relates to a microorganism of the genus Eremothecium which is produced by the process according to the invention.

Likewise, the invention relates to primary metabolites or end products of the energy metabolism, for example ethanol, acetate, lactate, acetone or butanol, inter alia, prepared in the above-described process according to the invention. In accordance with the invention, ethanol is produced at flow rates ranging from 0.06 to 0.40 h⁻¹, preferably from 0.09 to 0.32 h⁻¹, especially preferably from 0.25 to 0.32 h⁻¹ (Table 1).

The present invention makes it possible, for the first time, to observe a Crabtree effect, i.e. the formation of ethanol from glucose in the presence of an excessive supply of oxygen (aerobic fermentation), for a microorganism with filamentous growth.

The present invention likewise relates to secondary metabolites, for example antibiotics or gibberellins, intermediary metabolites, for example amino acids, citric acid or vitamins, inter alia, energy reserve materials, for example liquids, polysaccharides such as, for example, glycogen, dextran or xanthan or polyhydroxybutyric acid, inter alia, and extracellular or intracellular enzymes, for example amylases, proteases, cellulases or β-galactosidase, inter alia, which are prepared by the process according to the invention.

The present invention furthermore relates to the use of the microorganism produced by the process according to the invention or of the abovementioned metabolites according to the invention in fields of the chemical industry, of pharmacy, medicine, the foodstuff and/or feedstuff industry, and of agriculture and/or crop protection. The present invention likewise relates to the use of the metabolites according to the invention for the preparation of means for treating diseases in the abovementioned fields.

The present invention is illustrated in greater detail by the examples which follow, but which are not limiting:

1. Chemicals

The chemicals used are from Merck KGaA, Darmstadt, Fluka Chemie AG, Switzerland, and Sigma Chemie, Munich.

2. Analytical Methods

The results of the parameters described hereinbelow and measured during the continuous fermentation are compiled in Table 1.

2.1 Riboflavin

To determine the riboflavin concentration in the culture, 500 μl of culture liquid were treated with 50 μl of lysing enzyme solution (50 mg/ml) and then incubated for 1 hour in an Eppendorf shaker at 30° C. to release the protoplasts of the cells, and the protoplasts were made to burst by subsequently adding 450 μl of distilled water. The homogenate was filtered (pore size: 0.2 μm, Gelman Sciences) and the riboflavin content in the filtrate was determined by HPLC (Merck). The following separation conditions were selected (Schmidt et al, Microbiology, 1996, 142:419-426).

Column: LiChrospher 100 RP-18 (5 μm) (Merck)

| | |
|---|---|
| Mobile phase: | 50 mM NaH₂PO₄, |
| | 1 mM tetramethylammonium chloride |
| | 12% (v/v) acetonitrile |
| | H₃PO₄ to pH 3 |
| Flow rate: | 1 ml/min |
| Elution: | isocratic |
| Detection: | 270 nm |

2.2 Glucose

For the assay, culture liquid was filtered (circular paper filter, Schleicher & Schuell), and the glucose content in the filtrate was determined by the UV method for assaying D-glucose (Roche Diagnostics). The method is based on the enzymatic conversion of D-glucose and ATP to D-glucose-6-phosphate and ADP (enzyme: hexokinase). The enzyme glucose-6-phosphate dehydrogenase oxidizes D-glucose-6-phosphate to give D-gluconate-6-phosphate. At the same time, NADP⁺ is converted into NADPH. The amount of the NADPH formed is equivalent to the amount of the D-glucose employed. NADPH formation was measured in a UV spectrometer (UV-160, Shimadzu) at 340 nm.

2.3 Biomass

To determine the dry biomass (DBM) content, 30 to 200 ml of culture liquid were filtered through dried and weighed circular paper filters (Schleicher & Schuell). The filters were washed with distilled water, dried at 110° C. to constant weight and subsequently weighed (Monschau N., PhD thesis, Heinrich Heine University, 1998).

2.4 Ethanol and Acetate

Ethanol and acetate were determined by gas chromatography (Schmidt, PhD thesis, Heinrich Heine University, 1996). As was the case in the glucose assay, the culture liquid was filtered (circular paper filter, Schleicher & Schuell) and the filtrate was used for analysis. 500 μl of an internal standard were added to 500 μl of filtrate. This internal standard contained a defined amount of methanol, on the basis of which deviations, for example of the injection volume, were corrected when evaluating the chromatogram. A sample of 500 μl of ethanol/acetate standard solution +500 μl of internal standard were used for calibration.

Internal standard: 1.6 g/l methanol in 0.2 M HCl

Ethanol/acetate standard: 0.81 g/l ethanol, 1.64 g/l sodium acetate (corresponds to 1.18 g/l acetate)

The assay was carried out using an HP 5890 Series 11, Hewlett Packard, at an oven temperature of 155° C. and using nitrogen as the carrier gas.

2.5 Carbon Dioxide in the Exhaust Air

The CO₂ content in the exhaust of the fermenter was assayed by an infrared spectroscopic method (Stanbury & Whitaker, Oxford: Pergamon Press, 1984). The infrared CO₂ analyzer URAS 10E from Hartmann & Braun was used. Before the analysis, it was necessary to cool and dry the gas stream. Calibration was effected in the form of a two-point calibration using argon (0% (v/v) CO₂) and a calibration gas containing 5% (v/v) CO₂.

2.6 Partial Oxygen Pressure in the Culture

The partial oxygen pressure (pO₂) was measured using a pO₂ electrode (Mettler Toledo); (Demain & Solomon, Manual of Industrial Microbiology and Biotechnology, Washington, D.C., 1986). The two-point calibration was carried out in a temperature-controlled medium at constant stirring speed (650 rpm) immediately before inoculation. For zeroing, the medium was degassed with argon until a stable electrode signal was attained. The maximum value (pO₂=100%) was set by gassing with compressed air until a constant electrode signal was attained.

2.7 pH of the Culture

The pH was measured using a combined pH electrode (pH single-rod electrode) from Ingold. Calibration was effected before autoclaving, using commercially available standard solutions (pH 4 and pH 7).

2.8 Detection of Intracellular Fat Droplets

Nile Red staining was carried out to detect fatty deposits in the hyphae (Stahmann et al., Appl. Microbiol. Biotechnol., 1994, 42: 121-127). This dye has good solubility in a hydrophobic environment, but not in aqueous medium, and displays pronounced fluorescence under hydrophobic conditions (for example lipid deposits). Excitation is performed at 450 to 500 nm, and the emission wavelength is >528 nm. To carry out the staining procedure, approx. 50 μl of Nile Red solution (1 mg of Nile Red in 1 ml of acetone) were added to 500 μl of culture liquid. The fluorescence of the dye trapped in the intracellular fat drops was verified under the fluorescence microscope (photo and fluorescence microscope, Zeiss).

3. Nutrient Media

Complete HA medium (Stahmann et al., Appl. Microbiol. Biotechnol., 1994, 42: 121-127)

| | | |
|---|---|---|
| D (+)-glucose monohydrate | 10 | g/l |
| Yeast extract (granulated) | 10 | g/l |
| Complete HA medium agar plates | | |
| D (+)-glucose monohydrate | 10 | g/l |
| Yeast extract (granulated) | 10 | g/l |
| Agar (granulated) | 20 | g/l |

Minimal mineral-salt medium (Monschau N., PhD thesis, Heinrich Heine University, 1998, modified)

Solution A (100×): KH₂PO₄ 200 g/l, pH 6.7 with 8 M KOH

| | | | |
|---|---|---|---|
| Solution B (10x): | NH₄Cl | 15 | g/l |
| | L-asparagine (monohydrate) | 5 | g/l |
| | NaCl | 2 | g/l |
| | MgSO₄ × 7H₂O | 4 | g/l |
| | MnSO₄ × H₂O | 0.5 | g/l |
| | CaCl₂ × 2H₂O | 0.4 | g/l |
| | Myo-inositol | 1 | g/l |
| | Nicotinamide | 2.5 | g/l |
| | D (+)-glucose monohydrate | 10 | g/l |
| | Yeast extract (granulated) | 1 | g/l |
| | Glycine | 15 | mM |

Solution A, which has a concentration of 100×, is only added at the end to avoid the precipitation of sparingly soluble compounds.

4. Culture Methods

4.1 Strain Maintenance and Preculture

The wild-type strain of Eremothecium gossypii ATCC 10895 was maintained on complete HA medium agar plates which were stored at 4° C. and inoculated to fresh plates every 14 days.

To prepare precultures, 500 ml shake flasks (Schott Glaswerke), which were equipped with 2 baffles in order to improve the introduction of oxygen, containing 100 ml of complete HA medium were used. The shake flasks, including the medium, were sterilized by autoclaving. They were inoculated with mycelium from the strain maintenance plate, which mycelium was comminuted intensively with the aid of glass beads (diameter 5 mm). The material was cultured overnight on a shaker at 30° C. and 120 rpm. To provide the inoculum, the preculture was homogenized immediately prior to inoculation of the fermenter or the shake-flask series, likewise with the aid of glass beads.

4.2 Culture in the Laboratory Fermenter

The LABFORS system from Infors was used for the culture experiments in the laboratory fermenter. The stirred glass vessel had a maximum working volume of 5 liters. A two-tier disk stirrer equipped with 6 blades ensured mixing of the culture. The contamination risk inherent in using, for example, a face seal, was reduced by employing a magnetic clutch. In addition, the fermenter was provided with four baffles. It was aerated with compressed air via an aeration tube; the aeration rate was adjustable. The pO₂ was controlled via variation of the stirrer speed by an integral control unit. The temperature of the culture was controlled via the twin jacket of the fermenter. To reduce the escape of volatile substances (for example ethanol) with the gas stream, an exhaust-air condenser (4° C.) was used. A rising pipe was used for sampling, and in the case of continuous operation for harvesting. The orifice of the tube for sampling was stored in ethanol (96% (v/v)). The harvesting section used during the continuous operation did not require additional sterility-maintaining measures owing to its length. A sample of the culture was checked at regular intervals under the microscope for any contaminations. If required, it was possible to add an antifoam with the aid of an infusion pump (PRECIDOR-TYP 5003, Infors AG, flow rate: 2≧0.4 ml/h).

The online readings for pO₂, pH, temperature and speed were recorded and stored using the data gathering software MEDUSA 1.2 (Institut für Biotechnologie [department of biotechnology] 2, Forschungszentrum Jülich GmbH), and the CO₂ values from the exhaust air analysis were gathered using a recorder.

4.3 Continuous Culture in the Laboratory Fermenter

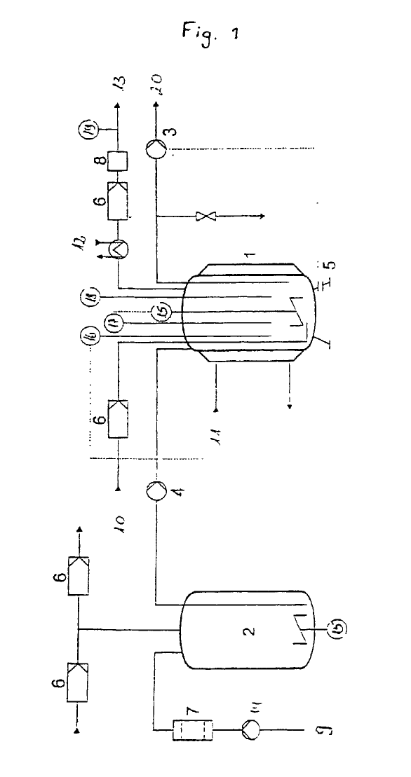

The experimental set-up for the continuous culture in the laboratory fermenter is shown in FIG. 1. In continuous operation, sterile nutrient medium was constantly pumped from the storage flask (2) (NALGENE, volume: 20 liters) into the fermenter (1) by means of a peristaltic pump (4) (Watson Marlow). The flow rate was adjusted by selecting the pumping capacity [ml/h]. The culture volume, more exactly the weight of the fermenter, was kept constant by the harvesting pump (3) (B. Braun AG), which was controlled by a balance (5) (Bioengineering AG), on which the fermenter rested. To have available a homogeneous medium, even when operating over prolonged periods, the storage container (2) was mixed by a magnetic stirrer (15).

The fermenter system, composed of the filtration section for the medium, the storage flask, the fermenter and the harvesting section, was sterilized by autoclaving. The medium was pumped into the storage flask (2) via the sterile filter (7) (SARTOBRAN-P CAPSULE, Sartorius AG), and the fermenter (1) was charged from the storage flask with 3 liters of medium. After all the electrode cables and tube connections had been connected and the reactor jacket filled completely, the balance (5) was tared and the weight to be kept constant was thus established. The fermenter was inoculated with approx. 90 ml of homogenized preculture. The culturing began in batch operation. After approximately 8 hours and a dry biomass content of approx. 1 g/l, the operation mode was switched to continuous, exploiting the advantageous physiological state of the culture during the exponential growth phase. The culture conditions were:

| | |
|---|---|
| Working volume: | 31 |
| Aeration: | 51/min |
| pO₂: | ≧80% (controlled via stirrer speed) |
| Temperature: | 28° C. |
| pH: | 6.7 |
| Stirrer speed: | 650 rpm ± 350 rpm |
| | (depending on the pO₂) |
| Nutrient medium: | minimal mineral-salt medium |

To establish the steady-state conditions, five residence times were allowed to pass, and at least 30 ml, at low biomass concentrations up to 200 ml, of culture liquid (20) were collected from the harvesting section for sampling, the sample container being stored on ice. Each equilibrium state was analyzed by reference to at least six readings at intervals of at least one hour.

Key to

FIG. 1: Schematic representation of the experimental set-up for the continuous culture of a microorganism of the genus Eremothecium in the laboratory fermenter

1: Fermenter

2: Medium storage container (sterile)

3: Harvesting pump

4: Peristaltic pump

5: Balance

6: Sterile filter (gas)

7: Sterile filter (liquid)

8: Cold trap

9: Medium (unsterile)

10: Compressed air: 5 l/min

11: Warm/cooling water

12: Cooling water: 4° C.

13: Exhaust air

14: Peristaltic pump

15: Magnetic stirrer

16: Temperature instrumentation

17: PO₂ instrumentation

18: pH instrumentation

19: CO₂ instrumentation

20: Culture liquid

FIG. 2: Graphic representation of the dry biomass (DBM) and riboflavin concentration curves of Eremothecium gossypii ATCC 10895 after lowering the flow rate (F) to 0.05 h⁻¹ from the steady state at a) 0.16 h⁻¹, b) 0.2 h⁻¹ and c) 0.3 h⁻¹ as a function of time (t).

TABLE 1
Overview over various parameters
measured during the continuous fermentation of
Eremothecium gossypii ATCC 10895

| | |
|---|---|
| F: | Flow rate [h⁻¹] |
| DBM: | Dry biomass [g/l] |
| n.d.: | Not detectable (<0.1 mg/l) |

TABLE 1

| F | DBM | Glucose | Riboflavin | Ethanol | Acetate |
|---|---|---|---|---|---|
| [h⁻¹] | [g/l] | [g/l] | [mg/l] | [g/l] | [g/l] |
| 0.05 | 2.88 | 0.07 | 2 | 0.28 | 0.05 |
| 0.09 | 2.50 | 1.10 | 1 | 0.89 | 0.21 |
| 0.10 | 1.84 | 2.52 | 1 | 0.89 | 0.22 |
| 0.20 | 1.47 | 4.24 | 1 | 0.98 | 0.28 |
| 0.25 | 1.18 | 4.60 | n.d. | 1.06 | 0.20 |
| 0.28 | 0.96 | 4.82 | n.d. | 1.16 | 0.19 |
| 0.32 | 0.91 | 4.95 | n.d. | 1.18 | 0.19 |
| 0.35 | 0.86 | 5.76 | n.d. | 0.78 | 0.22 |
| 0.41 | 0.54 | 7.58 | n.d. | 0.13 | 0.20 |
| 0.45 | 0.56 | 7.65 | n.d. | 0.10 | 0.21 |
| 0.50 | 0.40 | 7.87 | n.d. | 0.07 | 0.18 |

We claim:

1. A process for culturing a microorganism of the genus Eremothecium, which comprises culturing the cells in a continuous fermentation at flow rates ranging from greater zero to 0.8 h⁻¹.

2. A process as claimed in claim 1, which is carried out at flow rates ranging from 0.001 to 0.8 h⁻¹.

3. A process as claimed in either of claims 1 and 2, which is carried out at flow rates ranging from 0.01 to 0.7 h⁻¹, preferably from 0.05 to 0.6 h⁻¹.

4. A process as claimed in either of claims 1 and 3, wherein the flow rate is alternatingly adjusted to constant higher or lower values at defined intervals.

5. A process as claimed in any of claims 1 to 4, wherein a maximum specific growth rate ranging from 0.5-0.6 h⁻¹, in particular of 0.55 h⁻¹, is achieved.

6. A process as claimed in any of claims 1 to 5, wherein the cells are stably cultured continuously over a period of several weeks, preferably 2 to 8 weeks, especially preferably 2 to 4 weeks, in particular more than 2.5 weeks (corresponding to more than 400 hours).

7. A process as claimed in any of claims 1 to 6, wherein the cells grow predominantly as single cells and/or spores develop at flow rates ranging from greater zero to 0.20 h⁻¹, preferably 0.01 to 0.20 h⁻¹.

8. A process as claimed in any of claims 1 to 7, wherein the cells are predominantly formed as a hyphate mycelium and/or mycelial pellets are formed at flow rates ranging from ≧0.20-0.70 h⁻¹.

9. A process as claimed in any of claims 1 to 8, wherein homologous and/or heterologous and/or heterogeneous gene products are formed.

10. A process as claimed in any of claims 1 to 9, wherein a fungus of the species Eremothecium gossypii is employed.

11. A process as claimed in any of claims 1 to 10, wherein a microorganism which is genetically modified over the wild type Eremothecium gossypii ATCC 10895 is employed.

12. A microorganism of the genus Eremothecium produced by a process as claimed in any of claims 1 to 11.

13. A primary metabolite produced by a process as claimed in any of claims 1 to 11.

14. A secondary metabolite produced by a process as claimed in any of claims 1 to 11.

15. An intermediary metabolite produced by a process as claimed in any of claims 1 to 11.

16. An energy reserve material produced by a process as claimed in any of claims 1 to 11.

17. An enzyme produced by a process as claimed in any of claims 1 to 11.

18. The use of the microorganism as claimed in claim 12 or of the metabolites as claimed in any of claims 13 to 17 in fields of the chemical industry, of pharmacy, medicine, the foodstuff and/or feedstuff industry, and of agriculture and/or crop protection.
